(19) United States
(12) Patent Application Publication
(10) Pub. No.: US 2023/0091855 A1
(43) Pub. Date: Mar. 23, 2023

(54) Leadless Cardiac Pacemaker Device Configured to Provide Intra-Cardiac Pacing

(71) Applicant: BIOTRONIK SE & Co. KG, Berlin (DE)

(72) Inventors: Madeline Anne Midgett, Portland, OR (US); R. Hollis Whittington, Portland, OR (US); Ravi Kiran Kondama Reddy, Portland, OR (US); Christopher Jones, Oregon City, OR (US); Shayan Guhaniyogi, Portland, OR (US)

(73) Assignee: BIOTRONIK SE & Co. KG, Berlin (DE)

(21) Appl. No.: 17/792,031

(22) PCT Filed: Dec. 21, 2020
(86) PCT No.: PCT/EP2020/087361

§ 371 Date: Jul. 11, 2022

Related U.S. Application Data
(60) Provisional application No. 62/960,172, filed on Jan. 13, 2020.

(30) Foreign Application Priority Data
Mar. 11, 2020 (EP) 20162417.8

(51) Int. Cl. A61N 1/375; A61N 1/37; A61N 1/368

(52) U.S. Cl. CPC A61N 1/3756; A61N 1/3704; A61N 1/368

(57) ABSTRACT

A leadless pacemaker device configured to provide for an intra-cardiac pacing comprises a housing; an electrode arrangement arranged on the housing and configured to receive electrical signals; and a processing circuitry enclosed in the housing and operatively connected to the electrode arrangement, wherein the processing circuitry comprises a first processing channel having a first gain for processing a first processing signal derived from electrical signals received via the electrode arrangement and a second processing channel having a second gain for processing a second processing signal derived from electrical signals received via the electrode arrangement, the second gain being higher than the first gain.

### CROSS-REFERENCE TO RELATED APPLICATIONS

This application is the United States National Phase under 35 U.S.C. § 371 of PCT International Patent Application No. PCT/EP2020/087361, filed on Dec. 21, 2020, which claims the benefit of European Patent Application No. 20162417.8, filed on Mar. 11, 2020 and U.S. Provisional Patent Application No. 62/960,172, filed Jan. 13, 2020, the disclosures of which are hereby incorporated by reference herein in their entireties.

### TECHNICAL FIELD

The instant invention generally relates to a leadless cardiac pacemaker device for providing an intra-cardiac pacing, in particular a ventricular pacing, specifically VDD pacing.

### BACKGROUND

In recent years, leadless pacemakers have received increasing attention. Leadless pacemakers, in contrast to pacemakers implanted subcutaneously using leads extending transvenously into the heart, avoid leads in that the pacemaker device itself is implanted into the heart, the pacemaker having the shape of a capsule for implantation into cardiac tissue, in particular the right ventricular wall of the right ventricle. Such leadless pacemakers exhibit the inherent advantage of not using leads, which can reduce risks for the patient involved with leads transvenously accessing the heart, such as the risk of pneumothorax, lead dislodgement, cardiac perforation, venous thrombosis and the like.

Leadless pacemakers may specifically be designed for implantation in the right ventricle and, in this case, during implant are placed in or on the right ventricular wall. A ventricular pacing may, for example, be indicated in case a dysfunction at the AV node occurs, but the sinus node function is intact and appropriate. In such a case, in particular, VDD pacing may be desired, involving a ventricular pacing with atrial tracking and hence requiring a sensing of atrial activity in order to pace the ventricle based on intrinsic atrial contractions.

VDD pacing is, in particular, motivated by patient hemodynamic benefits of atrioventricular (AV) synchrony by utilizing an appropriate sinus node function to trigger ventricular pacing, potentially allowing to maximize ventricular preload, to limit AV valve regurgitation, to maintain low mean atrial pressure, and to regulate autonomic and neurohumoral reflexes.

Whereas, to the applicant's knowledge, VDD intra-cardiac leadless pacemakers are not currently available in the market, publications have explored solutions to use modalities to detect mechanical events of atrial contractions, including the sensing of motion, sound and pressure (see, for example, U.S. Publication No. 2018/0021581 A1 disclosing a leadless cardiac pacemaker including a pressure sensor and/or an accelerometer to determine an atrial contraction timing). As mechanical events created by atrial contraction generally exhibit a small signal volume when sensed from the ventricle, signals based on mechanical events, for example, motion, sound or pressure, may be difficult to detect. In addition, wall motion and movement of blood generated by atrial contractions may not be directly translated to the ventricle, and cardiac hemodynamic signals, such as motion, heart sounds and pressure, are likely affected by external factors such as posture and patient activity.

The present disclosure is directed toward overcoming one or more of the above-mentioned problems, though not necessarily limited to embodiments that do.

### SUMMARY

It is an objective to provide a leadless pacemaker device and a method for operating a leadless pacemaker device allowing, in particular, for ventricular pacing with atrioventricular synchrony, hence requiring a reliable sensing of atrial events in order to provide for ventricular pacing based on such atrial events.

Such desires are addressed by a leadless cardiac pacemaker device configured to provide for an intra-cardiac pacing and having the features of claim 1.

In one aspect, the pacemaker device comprises a housing; an electrode arrangement arranged on the housing and configured to receive electrical signals; and a processing circuitry enclosed in the housing and operatively connected to the electrode arrangement, wherein the processing circuitry comprises a first processing channel having a first gain for processing a first processing signal derived from electrical signals received via the electrode arrangement and a second processing channel having a second gain for processing a second processing signal derived from electrical signals received via the electrode arrangement. According to an embodiment, the second gain is higher than the first gain.

The leadless pacemaker device is configured to utilize multiple processing signals. For obtaining such processing signals, an electrode arrangement is provided, the electrode arrangement comprising of one or multiple electrodes to receive electrical signals from which the processing signals are derived. The processing signals herein, for example, may be obtained each using a pair of electrodes, wherein for obtaining the different processing signals the same pair of electrodes or different pairs of electrodes may be used. In the first case, a single electrical signal, such as an intra-cardiac electrogram, may be obtained, from which different processing signals, namely the first processing signal and the second processing signal are derived for separate processing. In a second case, separate electrical signals relating, for example, to a ventricular sensing signal and an atrial sensing signal (i.e., by applying a sensing optimized for atrial sensing) may be received in order to derive the first processing signal and the second processing signal from such different electrical signals, the different electrical signals, for example, being received using the same pair of electrodes or different pairs of electrodes of the electrode arrangement. In the latter case, the separate electrical signals are, for example, processed in different processing channels of the processing circuitry, the different processing channels associated with different sensing circuitry. For instance, one processing channel is configured as low gain processing channel for processing the electrical signal characterizing ventricular activity, while another processing channel is configured as high gain processing channel for processing the electrical signal characterizing atrial activity.

The different processing signals are processed in different processing paths of the processing circuitry. For this, the processing circuitry comprises a first processing channel for processing the first processing signal, the first processing signal relating, for example, to a near-field (in particular, ventricular) sensing signal which, according to the placement of the leadless pacemaker device, for example, in a ventricle of a patient's heart, may be strong such that the first processing channel may exhibit a rather low gain. In addition, the processing circuitry comprises a second processing channel for processing the second processing signal, which may relate, for example, to a far-field (e.g., atrial) sensing signal which, in case of a placement of the leadless pacemaker device, e.g., in the ventricle, may have a small amplitude, due to the distance between the location of implantation and the source of origin of the signals. In order to allow for a reliable processing of the second processing signal, the second processing channel exhibits a gain higher than the gain of the first processing channel, such that features relating to an atrial activity may be suitably analyzed within the received signals.

Because, for a placement of the leadless pacemaker device in, for example, the ventricle, atrial activity occurs in the far-field, atrial events within a regular ventricular sensing signal (for example, obtained via a regular ventricular QRS sensing channel) may be hard to discern, as a P wave stemming from atrial activity may exhibit a small amplitude in relation to QRS and T waves. For this reason signal portions relating to far-field activity may be processed separately from signals relating to near-field activity within the second processing channel, such that within the second processing channel far-field events may be detected with increased reliability and enhanced timing precision.

The housing provides for a hermetic sealing of the leadless pacemaker device, the leadless pacemaker device including all required components for autarkic operation, such as the processing circuitry, an energy storage such as a battery, electric and electronic circuitry and the like, within the housing. The housing is fluid-tight such that the leadless pacemaker device may be implanted into cardiac tissue and may be kept in cardiac tissue over an extended period of time to provide continuous cardiac pacing operation.

The leadless pacemaker device, in one aspect, is to be placed in the right or left ventricle.

In one aspect, the leadless pacemaker device comprises a fixation device having at least one fixation element arranged at the tip of the housing for fixing the pacemaker device to intra-cardiac tissue, in particular, the ventricular wall. In one embodiment, one or multiple fixation elements in the shape of thin wires, for example, Nitinol tines exhibiting a shape memory effect, may be provided. In another embodiment, a fixation element in the shape of a screw anchor may be provided.

In one aspect, the electrode arrangement comprises a first electrode arranged in the vicinity of a tip of the housing. The first electrode shall come to rest on cardiac tissue in an implanted state of the pacemaker device, such that the first electrode contacts cardiac tissue at a location effective for injecting a stimulating signal into cardiac tissue for provoking a pacing action, in particular, a ventricular pacing.

In one aspect, the electrode arrangement comprises a second electrode formed by an electrode ring circumferentially extending about the housing. Alternatively, the second electrode may, for example, be formed by a patch or another electrically conductive area formed on the housing. The second electrode is placed at a distance from the tip of the housing and hence at a distance from the first electrode arranged at the tip.

In one embodiment, the processing circuitry is configured to process, as said first processing signal, a first signal sensed between the first electrode and the second electrode. Such first signal may be denoted as near-field vector to be received between a pair of electrodes comprised of the first electrode and the second electrode. As the first electrode and the second electrode may, in one embodiment, be located at a rather close distance to each other, such pair of electrodes is predominantly suited to receive signals in close proximity to the leadless pacemaker device, i.e., in the near-field region within the ventricle if the leadless pacemaker device is implanted into the ventricle. The sense signal received in between the first electrode and the second electrode is provided to the first processing channel for processing in order to, for example, detect near-field (e.g., ventricular) events in the signal.

In one embodiment, the housing comprises a far end opposite the tip, the electrode arrangement comprising a third electrode arranged on the housing at the far end opposite the tip. The third electrode is operatively connected to the processing circuitry, such that the processing circuitry is enabled to receive and process signals received via the third electrode.

In one aspect, the processing circuitry is configured to process, as said second processing signal, a second signal sensed between the first electrode and the second or the third electrode. Such second signal vector arising between the first electrode and the second or the third electrode may be referred to as far-field vector, the first electrode and the third electrode exhibiting a distance with respect to each other larger than the first and the second electrode. The second signal may, in particular, be processed to detect events in the far-field, i.e., atrial contractions in case the leadless pacemaker device is placed in the ventricle, such that by means of the second signal an intrinsic atrial activity prior to injecting a pacing stimulus may be captured.

The second signal sensed between the first electrode and the third electrode may be used to sense intrinsic atrial contractions in order to provide for an atrial to ventricular synchronization by timely injecting a stimulus at the ventricular location of implantation of the pacemaker device following atrial contractions. The second signal is provided to the second processing channel in order to process the signal and detect atrial events from the signal, in order to provide for a pacing action based on detected atrial events, hence allowing for a ventricular pacing under atrioventricular (AV) synchrony.

In one aspect, the same set (or sub-set) of electrodes of the leadless pacemaker device is used both for sensing contraction signals as well as for emitting pacing stimulation signals. For this, in one embodiment, the processing circuitry of the leadless pacemaker device is configured to switch between a sensing mode and a stimulus mode by alternating between the processing of received signals and the generation of pacing signals. In particular, the processing circuitry may be configured to, in a first phase of the cardiac cycle, sense for atrial contractions. If atrial contractions are detected, the processing circuitry may sense ventricular activity within a time delay after the sensed atrial event. If no ventricular activity is sensed, the pacemaker device may switch to a stimulus mode in which a pacing signal is generated and emitted using at least one of the first electrode and the second electrode. After the pacing signal has been emitted, atrial contractions may be sensed anew to continue pacing operation.

In one embodiment, the second processing channel comprises a processing stage for differentiating one wave portion from another wave portion in the second processing signal. The processing stage, in particular, may be configured to apply, to the second processing signal, may include bandpass filtering, a blanking window for excluding a portion of the second reception signal from further processing, a moving average filtering, and a rectification. By means of the processing stage, in particular, such wave portions shall be isolated and/or emphasized within the signal to be processed which may be indicative of, e.g., an atrial event. If the leadless pacemaker device is placed in the ventricle of a patient's heart, signal portions relating to far-field (e.g., atrial) activity may have a much smaller amplitude than signal portions relating to a near-field (e.g., ventricular) activity. Hence, the processing serves to differentiate between the different signal portions in order to identify such signal portion which may contain signals relating to far-field (atrial) activity, namely the so-called P wave.

For isolating the P wave, a bandpass filtering may be applied, hence differentiating wave portions relating to the P wave from wave portions, in particular, relating to QRS and T waves stemming from ventricular activity. Alternatively or in addition, a blanking method may be applied in order to blank out certain portions of the second processing signal, namely such portions which contain signals stemming from events other than a far-field (e.g., atrial) activity. A blanking window, for this, serves to silence signal portions which are not of interest for far-field activity, but which may rather interfere with the detection of far-field activity. By means of a blanking window such portions of the signal which do not relate to far-field (e.g., atrial) activity hence are excluded from processing, such that the processing is limited to those signal portions (likely) relating to far-field activity. Alternatively or in addition, other methods such as a moving average filtering, finite differences or a rectification of the signal may be applied. A moving averaging filter herein can be used to smooth the processing signal. Rectification can serve to easily compare the processed signal to a (single) threshold in order to identify when the signal magnitude exceeds a predefined threshold. The rectified processed signal can also be compared to an adaptable threshold.

In one embodiment, the first processing channel comprises a first detection stage for detecting at least one near-field event in the first reception signal. Herein, the processing stage of the second processing channel may be configured to determine at least one limit of a blanking window for excluding a portion of the second reception signal from further processing based on a near-field (e.g., ventricular) event detected by the first detection stage of the first processing channel. The first processing channel serves to process a processing signal at a lower gain for detecting near-field events, i.e., events that are due to an activity in close proximity to the implanted leadless pacemaker device, for example, in the ventricle in which the leadless pacemaker device is implanted. As the near-field event will also be picked up in the second processing signal, it is advantageous to blank out such a signal portions relating to a near-field activity, i.e., QRS and T waves in in case of a placement of the leadless pacemaker device in the ventricle. In order to correctly locate the blanking window, detected near-field events may be taken into account, in order to, for example, determine a regular timing in between atrial events and ventricular events. From detected near-field events it may be determined in what time range after a far-field event a near-field event typically should occur, such that the blanking window, defined by a start time and a stop at time, may be appropriately set to blank out such portions of the second processing signal which relates to the near-field (ventricular) events.

During the blanking window, the second processing channel may, at least partially, be switched off in order to, for example, save power within the second, high gain processing channel. The second processing channel may, for example, comprise an amplification stage for amplifying the second processing signal, wherein the amplification stage may be switched off during the period of the blanking window such that that no power is consumed by amplification during the time interval of the blanking window.

A detection of far-field (atrial) events takes place in a detection window outside a blanking window. The detection window herein may start (immediately) at the end of a prior blanking window and may end at the beginning of the next blanking window. It, however, is also conceivable that the detection window, for example, starts at some time delay after the end of a prior blanking window. In between the end of the blanking window and the start of the detection window the second processing channel may be fully functional and process the associated second processing signal, wherein however a detection of far-field (atrial) events does not take place until the start of the detection window.

In one embodiment, the second processing channel comprises a second detection stage for detecting at least one far-field event in the second processing signal. The second detection stage may be arranged logically behind the processing stage of the second processing channel, such that the second detection stage receives processed signals from the processing stage of the second processing channel. The second detection stage herein serves to identify far-field (atrial) events in the second processing signal in order to output information relating to the timing of a detected far-field event.

The second detection stage of the second processing channel, in particular, may be configured to detect an atrial event by comparing the second processing signal to a threshold. In case the magnitude of the second processing signal exceeds a threshold, it may be concluded that a far-field event is present. The processing herein may take place on a rectified signal, which makes it possible to apply a single threshold to which the rectified signal may be compared. It, however, is also possible to identify a far-field (atrial) event in a non-rectified signal, for example, by applying two thresholds, namely a positive threshold and a negative threshold, wherein a far-field (atrial) event is identified if the positive signal portion exceeds the positive threshold and/or the (magnitude of the) negative signal portion exceeds the negative threshold.

The threshold(s) herein may be adaptable. For example, the threshold(s) may be set based on a maximum and/or minimum of a detected P wave. The threshold(s) may, for example, be set according to a predefined percentage of a maximum or minimum of a prior detected P wave.

Alternatively or in addition, one or multiple morphological features of the second processing signal may be evaluated in order to confirm or identify a far-field (atrial) event. The evaluation of one or multiple morphological features herein may include the determining of an amplitude value, the determining of a duration of a wave portion, for example, of a P wave, the determining of a number of threshold crossings or zero crossings, and/or the determining of a location of a wave portion in a detection window.

Generally, the identification of a far-field (atrial) event by comparing the second processing signal to one or multiple thresholds within the second processing channel allows for a fast detection and identification of a far-field (atrial) event. However, such detection possibly may not be fully accurate, due to, for example, a limited signal-to-noise ratio of far-field signal portions. In order to, hence, allow for a fast, yet accurate detection and identification of atrial events, it may be beneficial to confirm far-field (atrial) events detected by comparison to a threshold by evaluating morphological features of the processing signal processed in the second processing channel. This allows to, for example, confirm true P waves and to reject false detections stemming from noise, QRS waves or T waves. As the evaluation of morphological features, for example, by determining the length of a wave form, potentially requires an evaluation within a certain time interval, the confirmation may come after some delay from an initial identification of a far-field (atrial) event detected by a threshold crossing.

Hence, a far-field (atrial) may be preliminary detected by a threshold crossing, causing, for example, an atrial sense marker to be output by the second processing channel, the atrial sense marker being canceled later if the evaluation according to morphological features does not confirm the atrial event. Alternatively, an atrial sense marker may be output by the second processing channel only after confirmation according to morphological features, at the expense, however, of a potential delay of the output of the atrial sense marker with respect to the actual occurrence of the P wave.

In one aspect, the processing circuitry is configured to trigger a pacing signal based on at least one detected far-field (e.g., atrial) event. The leadless pacemaker device may provide for a synchronous pacing. For this, if no intrinsic ventricular activity is detected via the first processing channel within a defined delay time window, a simulation signal may be emitted by the pacemaker device for stimulating ventricular activity.

In one embodiment, in the event of loss of far-field (atrial) tracking due to, for example, a non-discernible contraction feature in the second processing signal, far-field tracking could temporarily not be based on the electrical signal received by means of the electrode arrangement, until a far-field tracking feature in the second processing signal is again identified. A pacing action (in particular for a ventricular pacing) could in this case be timed using an alternate atrial sensor, such as a motion sensor, impedance signal, or another indicator of atrial activity. Alternatively, the pacing could be switched to an asynchronous pacing mode. When a far-field activity cannot be tracked, the second processing channel may process the second processing signal in order to monitor continuously or at periodic intervals to determine when far-field events return in a possibly adaptive detection window.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein,

FIG. 1 shows a schematic view of the human heart;

FIG. 2 shows a schematic view of a leadless pacemaker device;

FIG. 3 shows a schematic view of a leadless pacemaker device, indicating signal vectors between different electrodes of the leadless pacemaker device;

FIG. 4 shows a schematic view of a processing circuitry of an embodiment of a leadless pacemaker device;

FIG. 5A shows a first processing signal in the shape of an intra-cardiac electrogram (IEGM) received by a first processing channel of the processing circuitry;

FIG. 5B shows a second processing signal, in a raw state prior to processing by a second processing channel;

FIG. 5C shows the second processing signal, after processing by means of a processing stage of the second processing channel; and

FIG. 5D shows the timing of detected ventricular and atrial events in the first processing signal and the second processing signal.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

In the instant invention, it is proposed to provide a leadless pacemaker device providing for an intra-cardiac pacing, in particular a ventricular pacing, specifically a so-called VDD pacing.

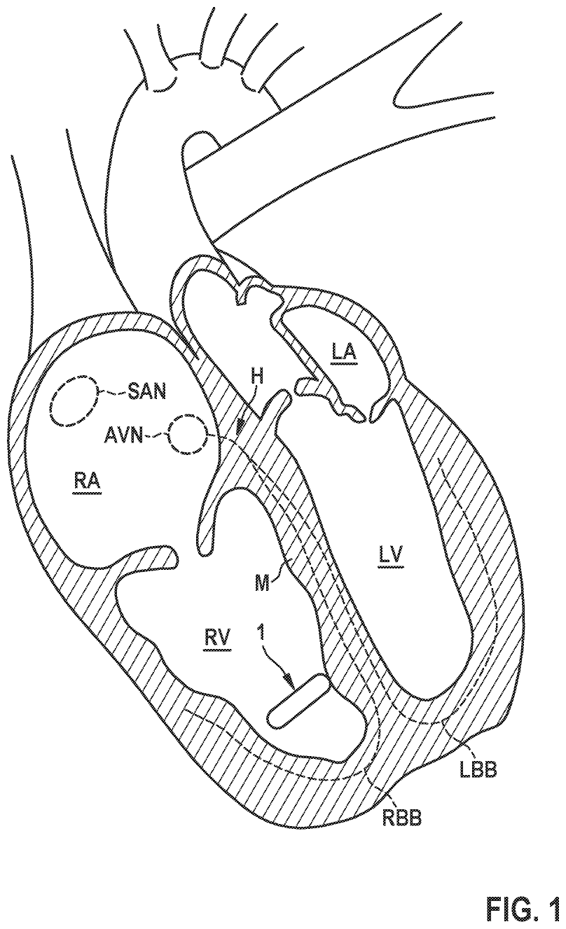

s FIG. 1 shows, in a schematic drawing, the human heart comprising the right atrium RA, the right ventricle RV, the left atrium LA and the left ventricle LV, the so-called sinoatrial node SAN being located in the wall of the right atrium RA, the sinoatrial node SAN being formed by a group of cells having the ability to spontaneously produce an electrical impulse that travels through the heart's electrical conduction system, thus causing the heart to contract in order to pump blood through the heart. The atrioventricular node AVN serves to coordinate electrical conduction in between the atria and the ventricles and is located at the lower back section of the intra-atrial septum near the opening of the coronary sinus. From the atrioventricular node AVN the so-called HIS bundle H is extending, the HIS bundle H being comprised of heart muscle cells specialized for electrical conduction and forming part of the electrical conduction system for transmitting electrical impulses from the atrioventricular node AVN via the so-called right bundle branch RBB around the right ventricle RV and via the left bundle branch LBB around the left ventricle LV.

In case of a block at the atrioventricular node AVN, the intrinsic electrical conduction system of the heart H may be disrupted, causing a potentially insufficient intrinsic stimulation of ventricular activity, i.e., insufficient or irregular contractions of the right and/or left ventricle RV, LV. In such a case, a pacing of ventricular activity by means of a pacemaker device may be indicated, such pacemaker device stimulating ventricular activity by injecting stimulation energy into intra-cardiac tissue, specifically myocardium M.

Within the instant text, it is proposed to use a leadless cardiac pacemaker device 1, as schematically indicated in FIG. 1, for providing for a ventricular pacing action.

Whereas common leadless pacemaker devices are designed to sense a ventricular activity by receiving electrical signals from the ventricle RV, LV they are placed in, it may be desirable to provide for a pacing action which achieves atrioventricular (AV) synchrony by providing a pacing in the ventricle in synchrony with an intrinsic atrial activity. For such pacing mode, also denoted as VDD pacing mode, it is required to sense atrial activity and identify atrial events relating to atrial contractions in order to base a ventricular pacing on such atrial events.

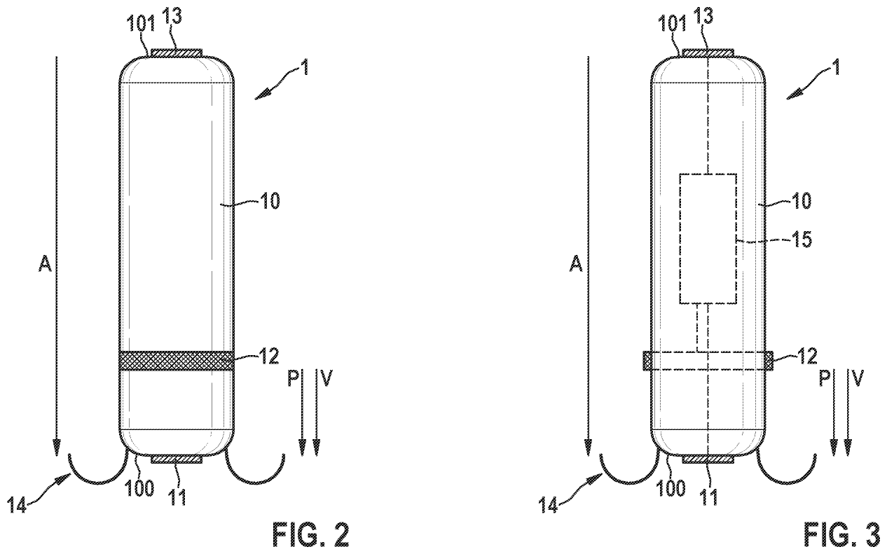

Referring now to FIGS. 2 and 3, in one embodiment, a leadless pacemaker device 1 configured to provide for an intra-cardiac pacing, in particular in a VDD pacing mode, comprises a housing 10 enclosing electrical and electronic components for operating the leadless pacemaker device 1. In particular, enclosed within the housing 10 is a processing circuitry 15, comprising, for example, also a communication interface for communicating with an external device, such as a programmer wand. In addition, electrical and electronic components such as an energy storage in the shape of a battery are confined in the housing 10. The housing 10 encloses components received therein, the housing 10 having the shape of, e.g., a cylindrical shaft having a length of, for example, a few centimeters.

The leadless pacemaker device 1 is to be implanted immediately on intra-cardiac tissue M. For this, the leadless pacemaker device 1 comprises, in the region of the tip 100, a fixation device 14, for example, in the shape of nitinol wires to engage with intra-cardiac tissue M for fixedly holding the leadless pacemaker device 1 on the tissue in an implanted state.

The leadless pacemaker device 1 does not comprise leads, but receives signals relating to a cardiac activity by means of an electrode arrangement arranged on the housing 10 and also emits stimulation signals by means of such electrode arrangement. In the embodiment of FIGS. 2 and 3, the leadless pacemaker device 1 comprises different electrodes 11, 12, 13 making up the electrode arrangement and serving to emit pacing signals towards intra-cardiac tissue M for providing a pacing and to sense electrical signals indicative of a cardiac activity, in particular, indicative of atrial and ventricular contractions.

A first electrode 11 herein is denoted as pacing electrode. The first electrode 11 is placed at a tip 100 of the housing 10 and is configured to engage with cardiac tissue M.

A second electrode 12 serves as a counter-electrode for the first electrode 11, a signal vector P arising between the first electrode 11 and the second electrode 12 providing for a pacing vector P for emitting pacing signals towards the intra-cardiac tissue M.

In addition, the second electrode 12 serves as a sensing electrode for sensing signals, in particular, relating to ventricular contractions, a signal vector V arising between the second electrode 12 and the first electrode 11, the signal vector V being denoted as near-field vector.

The second electrode 12 is placed at a distance from the first electrode 11 and, for example, has the shape of a ring extending circumferentially about the housing 10. The second electrode 12 is, for example, placed at a distance of about 1 cm from the tip 100 of the housing 10 at which the first electrode 11 is placed.

The leadless pacemaker device 1, in the embodiment of FIGS. 2 and 3, in addition comprises a third electrode 13 placed at a far end 101 of the housing 10, the third electrode 13 serving as a sensing electrode for sensing signals indicative of cardiac activity in the far-field. In particular, a signal vector A arises between the third electrode 13 and the first electrode 11, the signal vector A picking up signals being indicative, for example, of atrial contractions and being denoted as far-field vector.

The electrodes 11, 12, 13 are in operative connection with the processing circuitry 15, the processing circuitry 15 being configured to cause the first electrode 11 and the second electrode 12 to emit a pacing signal for providing a stimulation at the ventricle. The processing circuitry 15 furthermore is configured to process signals received via the electrodes 11, 12, 13 to provide for a sensing of cardiac activity, in particular, atrial and ventricular contractions.

In order to provide for a pacing in the ventricle in which the leadless pacemaker device 1 is placed, in particular to enable a pacing in the VDD mode, a sensing of atrial activity is required to provide for detected atrial sense markers in order to time a pacing in the ventricle to obtain atrioventricular (AV) synchrony. For this, a far-field signal from, in particular, the right atrium RA (see FIG. 1) shall be sensed in order to allow for a synchronous pacing in the right ventricle RV by means of the leadless pacemaker device 1 being implanted on intra-cardiac tissue M in the right ventricle RV.

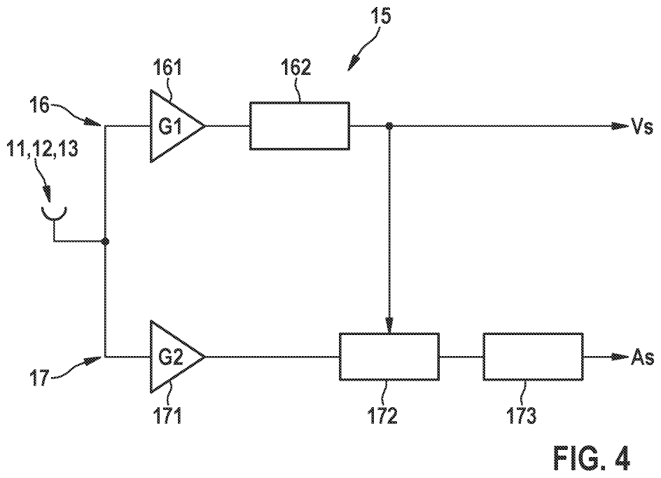

s Referring now to FIG. 4, the processing circuitry 15 comprises, in one embodiment, two processing channels 16, 17 for processing different processing signals relating to ventricular activity and atrial activity. Herein, typically, an intra-cardiac electrogram (IEGM) contains a signal portions relating to ventricular activity (in particular a QRS wave) and atrial activity (in particular a P wave), signal portions relating to atrial activity however resulting from a far-field signal source and hence being far less pronounced and having a far smaller amplitude then signal portions relating to a ventricular activity in the near-field, i.e., arising in close proximity to the implanted leadless pacemaker device 1. For this reason, the two processing channels 16, 17 are associated with different gains G1, G2, a first processing channel 16 serving to process a first processing signal to identify ventricular events at a rather low gain G1 and a second processing channel 17 being configured to process a second processing signal to identify atrial events at a significantly higher gain G2.

In particular, the first processing channel 16 is connected to the electrode arrangement comprised of the electrodes 11, 12, 13, the first processing channel 16 being configured, in particular, to sense and process a signal received via the electrodes 11, 12 (near-field vector V in FIGS. 2 and 3). The first processing channel 16 comprises a first amplification stage 161 having a gain G1 and, following the amplification stage 161, a detection stage 162 which is configured to identify ventricular sense markers Vs from the first processing signal processed within the first processing channel 16.

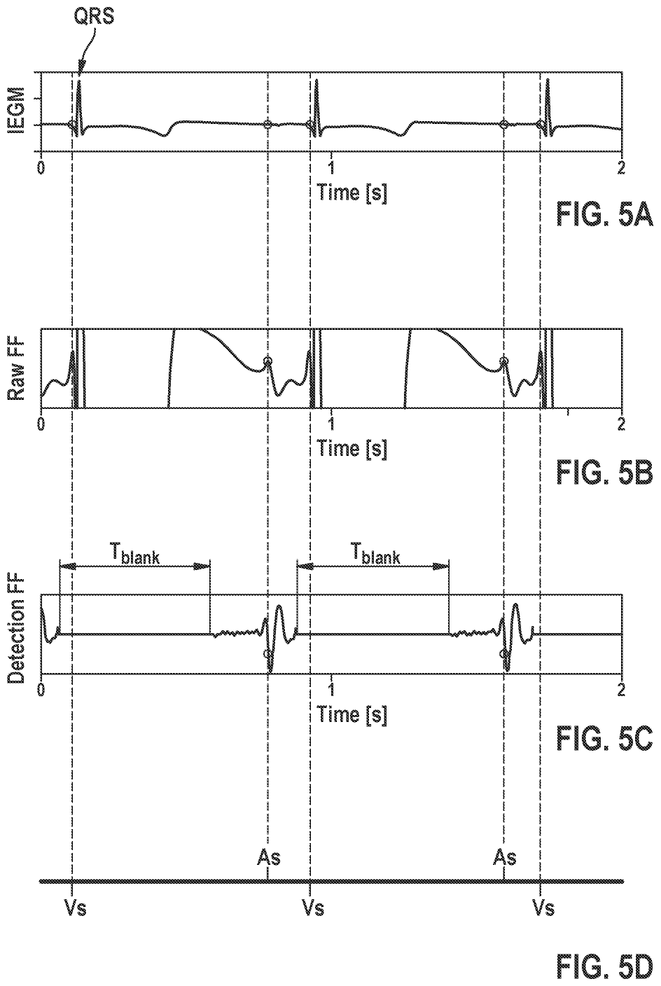

This is illustrated in FIG. 5A. The first processing signal processed within the first processing channel 16 resembles an intra-cardiac electrogram (IEGM) picked up in the ventricle in which the leadless pacemaker device 1 is implanted, the signal including portions relating to ventricular activity as well as atrial activity, signal portions relating to a ventricular activity however being far pronounced with respect to other signal portions (QRS waves as visible from FIG. 5A). For example, by means of comparing the signal to a suitable threshold or by applying a morphological analysis, the occurrence of ventricular events, in particular the start of a QRS wave, may be discerned, the second processing channel 16 outputting ventricular sense markers Vs indicative of such ventricular events.

The second processing channel 17 is likewise connected to the electrode arrangement comprised of electrodes 11, 12, 13, wherein the second processing channel 17 may, in particular, be configured to process a signal sensed via the far-field vector A, that is in between the electrodes 11, 13 placed at the tip 100 and the far end 101 of the housing 10 as illustrated in FIGS. 2 and 3. The second processing channel 17 comprises a second amplification stage 171 having a second gain G2, the second amplification stage 171 being followed by a processing stage 172 and a second detection stage 173.

The processing stage 172 serves to pre-process the second processing signal after amplification. The detection stage 173 in turn serves to evaluate and analyze the processed signal in order to identify atrial events within the second processing signal, the second processing channel 17 then outputting atrial sense markers As indicative of atrial events detected in the processed signal.

In order to identify and analyze atrial events, the gain G2 of the second processing channel 17 could be (significantly) higher than the gain G1 of the first processing channel 16. As visible from FIG. 5B illustrating the second processing signal in a raw state prior to processing by means of the processing stage 172, this allows to analyze signal portions relating to atrial events, but makes it necessary to discern such signal portions relating to atrial events from other signal portions, in particular, signal portions relating to ventricular events in the near-field and hence being far stronger than signal portions originating from atrial events in the far-field.

Within the processing stage 172, for example, a bandpass filtering, a partial blanking, a smoothing by means of a moving average filtering and a rectification may take place. A first or second order difference may be applied to remove a non-zero baseline while enhancing P wave detections.

FIG. 5C illustrates the signal after processing by means of the processing stage 172 in the second processing channel 17. In particular, within the processing a blanking window T_{blank} may be applied serving to mute the signal in such time intervals which—likely—do not relate to an atrial activity. The position of the blanking window T_{blank} herein is determined, in the embodiment of FIG. 4, according to an identification of prior ventricular events by means of the first processing channel 16.

In particular, by means of the detection of ventricular events in the first processing channel 16 a timing in between atrial events and ventricular events may be determined. According to such timing, a start point and an end point of the blanking window T_{blank} may be set, hence excluding signal portions from the processing which do not relate to atrial activity. Strong ventricular signals in this way may be suppressed such that signal portions relating to a ventricular activity may not interfere with a detection of atrial events.

During the blanking window T_{blank}, the second processing channel 17 may be turned off. In particular, the amplification stage 171 of the second processing channel 17 may be switched of in order to save power.

Generally, a detection for atrial events takes place outside of the blanking window T_{blank}. Herein, a detection window for detecting atrial events may start at the end of a prior blanking window T_{blank}. Alternatively, a detection window may have a delay with respect to the end of a prior blanking window T_{blank}, such that a signal processing within the second processing channel 17 starts at the end of a prior blanking window T_{blank}, a detection for atrial events however starting only after a certain delay.

For detecting atrial events within the second processing signal processed in the second processing channel 17 in order to output atrial sense markers As illustrated in FIG. 5D, different measures may be taken.

In order to allow for a fast detection of atrial events, the signal may, for example, be rectified within the processing stage 172, the detection stage 173 being configured to monitor whether the processed signal exceeds a (positive) threshold. Alternatively, no rectification may be applied in the processing stage 172, the detection stage 173 monitoring for a crossing of a positive threshold or a negative threshold (or both).

Such thresholds herein may be continuously adapted according to a detected P wave amplitude. In particular, the thresholds for a following cycle for a subsequent atrial event detection may be set according to a predefined percentage of the amplitude of a detected P wave in a prior cycle.

By monitoring a threshold crossing, a fast detection of atrial events is possible, however at the expense of a potentially reduced reliability. Therefore, in one embodiment, a further analysis of, for example, morphological features of the signals, in particular a detected P wave, may be applied in order to confirm an atrial event or, in the negative, to reject a false detection of an atrial event. For such morphological analysis, for example, the amplitude of a detected P wave, the duration of a detected P wave, a number of threshold crossings or zero crossings in a detected P wave, and/or a location of a detected P wave in the detection window may be evaluated. In case it is concluded from the morphological analysis that a true atrial event is present, the atrial event identified by the initial threshold crossing monitoring is confirmed. Otherwise, the atrial event is rejected as false.

As the morphological analysis requires an evaluation of the signal within a certain time interval, confirmation or rejection by means of the morphological analysis may come at a delay with respect to the identification according to a threshold crossing. It herein is possible to output an atrial sense marker. As already at the initial identification by means of a threshold crossing in order to then, after the morphological analysis, cancel the atrial sense markers in case of a false detection. Alternatively, an atrial sense marker As may only be output after a confirmation by means of the morphological analysis, at the expense of an increased delay in between a true occurrence of a P wave and the output of the atrial sense marker As.

Using atrial sense markers As output by the second processing channel 17, a ventricular synchronous pacing may be achieved. For this, it can be detected whether, following a detected atrial sense marker As, an intrinsic ventricular sense marker Vs occurs (output by the first processing channel 16) within a predefined time delay window after the atrial sense marker As, in which case no stimulation is required. If no ventricular sense marker Vs is detected, a stimulation pulse may be emitted, causing a synchronous pacing at the ventricle.

Conversely, also an asynchronous pacing can be performed.

Utilizing a far-field electrical signal received by means of a leadless pacemaker device can offer a superior detection of far-field events, in particular atrial events in case the leadless pacemaker device is implanted into the ventricle. A tracking of far-field events by using and evaluating electrical signals may allow for an increased consistency and reliability, in particular with respect to external factors such as posture and patient activity.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Leadless pacemaker device
10 Housing
100 Tip
101 Far end
11 First electrode (pacing electrode)
12 Second electrode (pacing ring)
13 Third electrode
14 Fixation device
15 Processing circuitry
16 Processing channel
160 Electrode arrangement
161 Amplification stage
162 Detection stage
17 Processing channel
170 Electrode arrangement
171 Amplification stage
172 Processing stage
173 Detection stage
A Atrial vector
As Atrial event
AVN Atrioventricular node
G1, G2 Gain
H HIS bundle
LA Left atrium
LBB Left bundle branch
LV Left ventricle
M Intra-cardiac tissue (myocardium)
P Pacing vector
RA Right atrium
RBB Right bundle branch
RV Right ventricle
SAN Sinoatrial node
T_{blank} Blanking window
V Ventricular vector
Vs Ventricular event

1. A leadless pacemaker device configured to provide for an intra-cardiac pacing, the leadless pacemaker device comprising:
a housing;
an electrode arrangement arranged on the housing and configured to receive electrical signals; and
a processing circuitry enclosed in the housing and operatively connected to the electrode arrangement, wherein the processing circuitry comprises a first processing channel having a first gain for processing a first processing signal derived from electrical signals received via the electrode arrangement and a second processing channel having a second gain for processing a second processing signal derived from electrical signals received via the electrode arrangement, the second gain being higher than the first gain.

2. The leadless pacemaker device according to claim 1, wherein the housing comprises a tip, the electrode arrangement comprising a first electrode placed on the tip for engaging with intra-cardiac tissue.

3. The leadless pacemaker device according to claim 2, wherein the electrode arrangement comprises a second electrode formed by an electrode ring circumferentially extending about the housing and being placed at a distance from said tip.

4. The leadless pacemaker device according to claim 3, wherein the processing circuitry is configured to process, as said first and/or second processing signal, a first and/or second signal sensed between the first electrode and the second electrode.

5. The leadless pacemaker device according to claim 2, wherein the housing comprises a far end opposite the tip the electrode arrangement comprising a third electrode arranged in the vicinity of the far end.

6. The leadless pacemaker device according to claim 5, wherein the processing circuitry is configured to process, as said first and/or second processing signal, a first and/or second signal sensed between the first electrode and the third electrode.

7. The leadless pacemaker device according to claim 1, wherein the processing circuitry is configured to process the first processing signal to detect a ventricular activity and the second processing signal to detect an atrial activity.

8. The leadless pacemaker device according to claim 1, wherein the second processing channel comprises a processing stage for differentiating one wave portion from another wave portion in the second processing signal.

9. The leadless pacemaker device to claim 8, wherein the processing stage configured to apply, to the second processing signal, at least one of a bandpass filtering, a blanking window (T_{blank}) for excluding a portion of the second processing signal from further processing, a moving average filtering, a finite difference filtering, and a rectification.

10. The leadless pacemaker device according to claim 8, wherein the first processing channel comprises a first detection stage for detecting at least one near-field event in the first processing signal, wherein the processing stage of the second processing channel is configured to determine at least one limit of a blanking window (T_{blank}) for excluding a portion of the second processing signal from further processing based on at least one near-field event detected by said first detection stage.

11. The leadless pacemaker device according to claim 10, wherein the second processing channel comprises an amplification stage for amplifying the second processing signal, wherein the amplification stage is switched off during said blanking window.

12. The leadless pacemaker device according to claim 1, wherein the second processing channel comprises a second detection stage for detecting at least one far-field event in the second processing signal.

13. The leadless pacemaker device according to claim 12, wherein the second detection stage is configured to detect said at least one far-field event by at least one of
comparing the second processing signal to a threshold, and evaluating at least one morphological feature of the second processing signal, wherein the evaluating of at least one morphological feature includes at least one of determining an amplitude value from the second processing signal,
determining a duration of a wave portion of the second processing signal,
determining a number of threshold crossings or zero crossings in the second processing signal, and
determining a location of a wave portion in a detection window.

14. The leadless pacemaker device according to claim 12, wherein the processing circuitry is configured to trigger a pacing signal based on at least one detected far-field event.

15. Method for operating a leadless pacemaker device for providing intra-cardiac pacing, comprising:
receiving electrical signals, using an electrode arrangement arranged on a housing of the leadless pacemaker device; and
processing, using a processing circuitry enclosed in the housing and operatively connected to the electrode arrangement, a first processing signal and a second processing signal derived from electrical signals received via the electrode arrangement, wherein the processing circuitry comprises a first processing channel having a first gain for processing said first processing signal and a second processing channel having a second gain for processing said second processing signal, the second gain being higher than the first gain.
